(19) United States
(12) Patent Application Publication
(10) Pub. No.: US 2007/0135915 A1
(43) Pub. Date: Jun. 14, 2007

(54) Implantable lens device

(76) Inventor: William L. Klima, Fredericksburg, VA (US)

Correspondence Address: KLIMA LAW OFFICES, P.L.L.C., P. O. Box 2855, Stafford, VA, 22555-2855, US

(21) Appl. No.: 11/656,960

(22) Filed: Jan. 24, 2007

Related U.S. Application Data
(63) Continuation-in-part of application No. 10/943,405, filed on Sep. 17, 2004.

(51) Int. Cl. A61F 2/16

(52) U.S. Cl. 623/6.37; 623/6.46

(57) ABSTRACT

An implantable lens device including a lens optic portion connected to a lens haptic portion. The implantable lens device, for example, is an accommodating intraocular lens device configured to provide accommodative movement of the lens optic portion within the eye.

### CONTINUING APPLICATION INFORMATION

This is a continuation in part of U.S. patent application entitled “INTRAOCULAR LENS DEVICE”, Ser. No. 10/943,405, filed on Sep. 17, 2004.

### FIELD OF THE INVENTION

The present invention is directed to an implantable lens device, in particular a reinforced and/or prestressed implantable lens device, for implantation in the eye preferably through a small incision in the eye. The implantable lens device according to the present invention, for example, can be an improved intraocular lens (IOL), phakic refractive lens, accommodating lens, accommodating intraocular lens, anterior chamber lens, posterior chamber lens, artificial ocular lens implant, and other artificial ocular implant.

### BACKGROUND OF THE INVENTION

There exist many types, designs or otherwise configurations of implantable lens devices, including intraocular lens devices, and more recently accommodating intraocular lens devices and phakic refractive lens devices.

Intraocular lens devices have been very successful for use in cataract surgery after the natural crystalline lens has been removed from the capsular bag located in the posterior chamber of the eye. More recently, implantable lens are being configured for refractive correction of the eye such as a refractive correction lens, phakic refractive lens (prl) or implantable contact lens (icl) configured to be implanted between the natural crystalline lens and the iris, or the artisan lens configured for implantation in the anterior chamber of the eye.

There exists a need for an implantable lens device configured to be adjustable, preferably in vivo after implantation in the eye, for example, to fine tune the fit, refractive power, shape of the lens portion, shape the haptic portion, or otherwise provide an adjustable configuration of the intraocular lens device. Further, there exists a need for an accommodating intraocular lens device configured to provide enhanced accommodation, or for providing an accommodation multiplier or amplifier.

Even further, there exists a need for a reinforced implantable lens device wherein the haptic(s) are reinforced or stiffened or strengthened, the lens optic portion, or wherein the entire implantable lens device is reinforced or stiffened (e.g. plate haptics only, lens portion only, entire lens device edge-to-edge). For example, reinforcing or strengthening or stiffening the haptic(s) and/or optic of an accommodating lens device can enhance accommodation. As a further example, reinforcing or stiffening a phakic refractive lens can allow the phakic refractive lens to maintain its shape or substantially maintain its shape even when forces are exerted on the phakic refractive lens. Alternatively, the phakic refractive lens is reinforced or stiffened so that the phakic refractive lens can flex or bend, however, having memory to return to its original shape.

### SUMMARY OF THE INVENTION

A first object of the present invention is to provide an improved implantable lens device.

A second object of the present invention is to provide an improved hingeless implantable lens device.

A third object of the present invention is to provide an improved hinged implantable lens device.

A fourth object of the present invention is to provide an improved intraocular lens device.

A fifth object of the present invention is to provide an improved accommodating lens device.

A sixth object of the present invention is to provide an improved phakic refractive lens device.

A seventh object of the present invention is to provide an adjustable implantable lens device configured to be adjusted in situ and/or in vivo within the eye.

An eigth object of the present invention is to provide a reinforced implantable lens device.

A ninth object of the present invention is to provide a pre-stressed implantable lens device.

A tenth object of the present invention is to provide an improved reinforced accommodating lens device.

An eleventh object of the present invention is to provide an improved accommodating lens device configured to accommodate by application of forces exerted on the haptic portion(s), in particular on a plate haptic portion(s) of the accommodating lens device.

A twelveth object of the present invention is to provide an improved accommodating lens device configured to accommodate by application of compression and/or tension on the haptic portion, in particular on a plate haptic portion of the accommodating lens device.

A thirteenth object of the present invention is to provide an improved accommodating lens device configured to accommodate by application of forces on the haptic portion(s) and a differential pressure on opposite sides of the lens optic portion.

A fourteenth object of the present invention is to provide an improved accommodating lens device configured to accommodate by application of tension on the accommodating lens device.

A fifteenth object of the present invention is to provide an improved accommodating lens device configured to accommodate by application of compression, tension, and/or shear on the accommodating lens device.

The present invention is directed to an implantable lens device, for example, an intraocular lens, an accommodating lens, an accommodating intraocular lens, a phakic refractive lens (prl), an anterior chamber lens, a posterior chamber lens, or other artificial ocular lens device.

In one preferred embodiment, the lens device is an adjustable in situ and/or in vivo, for example, with electromagnetic radiation. For example, the lens device is pre-stressed to allow for a change of size, shape, configuration, orientation, or change of chemical properties and/or physical properties of the lens device in situ (prior or after implantation), in situ and in vivo (indirectly radiated after implantation) and/or in vivo (directly radiated after implantation).

A preferred embodiment of the intraocular lens device according to the present invention is configured to be adjustable, preferably in situ and in vivo, after implantation into the eye. Specifically an implantable lens device according to the present invention can be a lens only, a lens portion in combination with looped type haptics, lens portion in combination with a plate type haptic, and a lens portion in combination with both a plate type haptic and a loop type haptic. A preferred embodiment utilizes a lens portion in combination with a plate haptic portion, for example, at least one pair of opposed plate haptic portions or a surrounding haptic portion.

The adjustable implantable lens device according to the present invention can be configured to be allow adjustment of one or more portions of the implantable lens device, preferably in vivo, after implantation in the eye. For example, the fit of the lens optic portion, the fit of the lens haptic portion, the fit of both the lens optic portion and the lens haptic portion, the size of the lens optic portion, the size of the lens haptic portion, the size of both the lens optic portion and the lens haptic portion, the shape of the lens optic portion, the shape of the lens haptic portion, the shape of both the lens optic portion and lens haptic portion, the lens surface properties (e.g. surface tension, surface energy, surface finish, molecular or atomic changes to the surface, surface chemistry, internal chemistry of a portion or all the lens device, color of surface, light transmittance of surface, light reflectance of surface, adherence properties of surface with tissue, lubrication properties of surface and lens, hydrophobic or hydrophilic properties of surface and lens, cross-linking of lens material internally or at surface, structural stiffness of lens optic portion, structural stiffness of lens haptic portion, structural stiffness of both lens optic portion and lens haptic portion, change in length, change in width, and/or change in thickness of lens optic portion, change of length, change of width and/or change of thickness of the lens haptic portion, change of length, change of width and/or change of thickness of both lens optic portion and lens haptic portion, color of lens optic portion, color of lens haptic portion, color of both lens optic portion and lens haptic portion, change of hardness of lens optic portion, change of hardness of lens haptic portion, change of hardness of both lens optic portion and lens haptic portion, change of symmetry of lens optic portion, change of symmetry of lens haptic portion, change of symmetry of both lens optic portion and lens haptic portion, aspheric correction of lens optic portion, toric correction of lens optic portion can be adjusted, preferably in vivo, after implantation of the implantable lens device in the eye.

The implantable lens device according to the present invention can be changed one time, changed many times a periodic on non-periodic time intervals (i.e. repeatedly), changed over a period of time (e.g. aging), change on demand, or changed intoother modes of functioning. Preferably the implantable lens device according to the present invention is adjusted by means of electromagnetic radiation applied to one or more portions of the implantable lens device. For example, electromagnetic radiation (e.g. heat, light, infrared, ultraviolet, laser, x-rays) can be applied into the eye from a source located outside of the eye, for example, through the cornea and iris of the eye. Further, the electromagnetic radiation source or transmitter can be located within the eye (e.g. emitting portion of instrument positioned within eye, for example, through a small incision) to emit radiation directly (i.e. source in close or direct contact with implanted intraocular lens device) or indirectly (i.e. source emits electromagnetic radiation through eye fluid into implanted intraocular lens device). Alternatively, the implantable lens device according to the present invention can be combined with an internal energy source (e.g. battery) and electromagnetic radiation emitter implanted within the eye and/or body of the patient. As a further example, electrical components can be provided within the implantable lens device, eye and/or body to change, convert, control, regulate or otherwise interface with an energy source located outside of the eye and/or body (e.g. energy transferred through cable or through portions of body or eye without incision, for example, by use of spinning magnets, radio wave transmission or other known methods of transferring energy through skin or tissue).

The implantable lens device according to the present invention can be configured from a single material used to make both the lens optic portion and lens haptic portion, or a combination of different materials. For example, the lens optic portion is preferably made from a deformable or resilient material (e.g. silicon, acrylic, collagen containing polymer, other known suitable biomaterial) and the lens haptic portion can be made from a resilient and/or deformable material having a higher tensile, compression, and/or shear strength (e.g. polyimide, polyester, polyamide). The lens optic portion and/or lens haptic portion can be configured to be adjustable by use of electromagnetic radiation in a variety of ways. For example, the implantable lens device can be adjusted by treating a specific points, axis, areas, surfaces, planes or volumes with electromagnetic radiation causing chemical reactions of material(s) of the implantable lens device, changing of physical properties of the implantable lens device, additional cross-linking of the polymer material of the implantable lens device, degradation of cross-linking of the polymer material, annealing, welding, heating, softening, hardening, loss of tensile strength, increase of tensile strength, decrease in hardness, increase in hardness, formation of bubbles, elimination of bubbles, formation of voids, elimination of voids, formation of tension, formation of compression, formation of shear, increased opacification, decreased opacification, increased light transmittance, decreased light transmittance, increased curvature, decreased curvature and many other chemical and physical characteristics, properties and/or features of the implantable lens device.

The implantable lens device can be provided with one or more points, particles, layers, surfaces, matrix, or inserts made of different material(s) versus the base lens device material to provide surfaces of interface therebetween. The electromagnetic radiation can be focused at the points, particles, layers, surfaces, matrix, inserts or interfaces to change the chemical and/or physical characteristics of the lens optic portion and/or lens haptic portion of the implanted lens device. The points, particles, layers, surfaces, matrix or inserts can be on an atomic or molecular scale (e.g. nano, micro size, or even macroscopic size). For example, the inserts can be made of different materials located in a specific matrix arrangement within the lens portion (e.g. monomers, polymers, metal atoms, metal complexes, salts, metal salts, inorganic molecules, or metal containing molecules can be used as additives or arranged in a particular arrangement (e.g. matrix) within the lens optic portion, which can further catalyzed cross-linking of polymer (i.e. chemically), or by simply heating up to increase the extent of cross-linking of the polymer. In some embodiments, for example, an interior portion or portions of the lens portion are soften or possibly liquefied by application of electromagnetic radiation focused at these interior portions to model a young highly accomodative natural crystalline lens. On a very sophisticated basis, layers of different hardness from the center of the lens optic portion outwardly can be formed by application of laser light by three-dimensional application thereof at interior locations within the lens optic portion.

Another preferred implantable lens device according to the present invention is a pre-stressed implantable lens device. Specifically, the implantable lens device is configured and/or manufactured in a manner so that the implantable lens device is in a pre-stressed condition after formation thereof. For example, a silicon deformable implantable lens device is made in a mold having a predetermined and pre-stressed configuration, and after removal of the deformable implantable lens device from the mold, the deformable implantable lens device changes configuration or conformation due to the pre-stressing thereof. In another embodiment, the implantable lens device retains its shaped after molding, but is pre-stressed during molding, and then the implantable lens device changes shape after being treated with electromagnetic radiation in situ and/or in vivo. Alternatively, an implantable lens device is made from a dehydrated lens blank, and is pre-stressed prior to, during, or after machining. The pre-stressed blank is machined and then after machining, the lens is hydrated and allowed to assume its new configuration or conformation. As an additional type of pre-stressing, the implantable lens device is provided with one or more reinforcements or inserts that are pre-stressed (e.g. tension and/or compression) before, during, and/or after the implantable lens device is being formed or made. For example, an insert is placed under tension in a mold while the implantable lens device is molded around the insert. After molding, the tension is relieved on the insert placing portions of the implantable lens device under compression. Alternatively, an insert is placed under compression while molding the implantable lens portion, and again the compression force is relieved after formation thereof.

A further embodiment of the implantable lens device according to the present invention is an accommodating lens device, in which the configuration or conformation of the intraocular lens is preferably changed in vivo, after implantation in the eye, by application of compression, tensile, and/or shear forces on the edges of the accommodating lens device and/or a pressure differential on opposite sides of the accommodating lens device. For example, the accommodating lens device is configured with the lens optic portion located in a different plane relative to the lens haptic portion, and when compression and/or tensile force(s) is applied to the edges of the accommodating lens device, the lens optic portion moves away or towards the plane containing the lens haptic portion(s) depending on the particular arrangement or design.

The above described preferred embodiments of the implantable lens device according to the present invention, include an adjustable implantable lens device, a pre-stressed implantable lens device, and an accommodating implantable lens device. These can be separate features of the implantable intraocular lens device, or can be utilized in various combinations.

Preferred embodiments of the implantable accommodating lens device according to the present invention are configured to move the lens optic portion of the accommodating lens device within the eye by compression forces, tension forces, pressure, pressure differential, or a combination thereof applied to the accommodating lens device. These forces can be exerted on the outer surface of the accommodating lens device. For example, the forces can be exerted on the lens haptic or lens haptic portions, particularly applied to the edges and surfaces of the lens haptic portion. Further, force(s), pressure(s), pressure differential(s) can be exerted on the lens optic portion to also cause movement of the lens optic portion within the eye.

A preferred embodiment of the implantable lens device according to the present invention is an accommodating lens device configured so that the accommodating lens device has a fixed configuration, which can be resiliently deformed or change shape by the application of force (e.g. compression, tension, pressure, pressure differential) on the accommodating lens device. Another preferred embodiment of the accommodating lens device according to the present invention is an accommodating lens device having memory so that it returns to its original shape or configuration when the application of force is relieved (i.e. elastic material deformation, not plastic deformation). Specifically, the accommodating lens device resiliently bends or resiliently distorts or deforms under force, however, the implantable lens device returns to its original shape after the force is relieved.

Preferred embodiments of the implantable lens device according to the present invention preferably utilize one or more reinforcement or reinforcement portions for increasing the compressive, tensile, and/or shear strength (e.g. stiffness) of one or more portions of the lens haptic portion(s) and/or lens optic portion. The reinforcement portion can be configured, made, devised, tailored, designed or otherwise specified to be soft, hard, stiff, deformable, non-deformable, resilient, or have other physical and/or chemical characteristics similar or different relative to the base lens material (i.e. the main base material percentagewise making up the haptic portion(s) or lens portion(s) or overall implantable lens device) while still reinforcing one or more portions of the implantable lens device. A preferred reinforcement according to the present invention is a resilient reinforcement that is still highly flexible, resilient, bendable, or otherwise deformable like the lens material so that the implantable lens device can still be implanted through a small incision, in particular with use of an lens injecting device or forceps.

The reinforcement or reinforcement portion according to the present invention can have greater compression, tensile and/or shear strength verses the lens material(s). The reinforcement(s) can be embedded within the lens material (e.g. when molding the implantable lens device) and/or provided on one or more surfaces of the lens haptic portion(s) and/or lens optic portion (e.g. embedding when molding the implantable lens device, or added after molding by chemical adhering or welding). The reinforcement(s) can be designed or devised to resist bending of one or more portions of the lens haptic portion(s) (e.g. devised to resist bending along length of the lens haptic portion(s)) and/or stiffening of the lens optic portion from bending (e.g. devised to prevent deforming or flexing or bending of the lens optic portion during use in the eye, but still deformable enough to undergo implantation through a small incision using a lens injector or forceps).

Alternatively, the reinforcement(s) can be devised to concentrate or even multiple force at one or more point(s), axis(es), plane(s), surface(s), location(s), space(s) or volume(s) within the lens haptic portion(s) and/or lens optic portion(s) (e.g. to enhance bending at a particular point(s), axis(es), plane(s), location(s), space(s) or volume(s) within the haptic portion(s) and/or lens portion(s). For example, a lens plate haptic or lens plate haptics can be reinforced to carry compressive, tensile, and/or shear forces exerted on the implantable lens device from the eye in a manner to enhance bending at a particular point(s), axis(es) particularly at, near or adjacent the physical connections between the lens plate haptic(s) and the lens optic portion in an accommodating lens device. This arrangement focuses or concentrates the forces specifically at these connections between the lens haptic portion(s) and the lens optic portion to enhance bending of the lens material at this location. These connections can be arranged or designed to purposely not reinforced the base lens material at these connections so that the full concentrated force is delivered via the reinforcement(s) to concentrate or even amplify or multiple, depending on mechanical design, the amount or degree of bending (i.e. providing a force concention, or even amplifier or multiply bending forces at these connection points) to enhance bending at the connection between the lens haptic portions and lens optic portion to enhance accommodation.

The implantable lens device according to the present invention can be made by a variety of different methods. For example, the lens base material can be molded and/or machined (e.g. using a mold, drill press, mill, lathe, CNC, grinding machine, polishing machine, or other known machining equipment). The reinforcement(s) can be molded, extruded, cut, cut from a sheet of material (e.g. cut with a high pressure waterject, shears, stamping tool, laser, chemically etched, radiation), or machined (e.g. using a drill press, mill, lathe, CNC, grinding machine, polishing machine, or other know machining equipment). The lens optic portion and lens haptic portion(s) can be made as a single piece or multiple pieces assembled together. The implantable lens device can be provided with the reinforcement(s) before, during or after making of the implantable lens device by molding and/or machining. Alternatively, the reinforcement(s) can be made separate from the implantable lens device, and then connected to or integrated into the implantable lens device (e.g. embedded, added as an insert, layered, mechanically connected, snap fit connected, adhered, cemented, welded, bonded, chemically bonded).

A particularly preferred embodiment of an accommodating lens device according to the present invention includes at least one set of opposed lens plate haptic portions connected to a lens optic portion. The lens plate haptic portions each include at least one separate reinforcement provided along the length dimension of each lens haptic portion so that the lens haptic portions are more rigid or less flexible along their lengths. The at least one reinforcement preferably ends before the point or axis of connection between the plate haptic portions and the lens optic portion. This arrangement enhances the extent or degree of bending of the lens plate haptic portions relative to the lens optic portion to enhance accommodative movement of the lens optic portion. In a more preferred embodiment, the reinforcement(s) is configured so that the accommodating lens device can be folded, rolled, compressed, or otherwise reduced in at least the width dimension to allow insertion through a small incision in the eye with forceps or a lens injector. For example, the lens plate haptics are reinforced by one (1), two (2), or three (2) parallel longitudinal reinforcement stringers. In another embodiment, the reinforcement(s) extends in two (2) dimensions (i.e. length and width), and is configured (e.g. highly flexible) so that the accommodating lens device can be reduced in both the length and width dimensions for insertion through a small incision in the eye.

In another embodiment of the implantable lens device according to the present invention, the overall implantable lens device is reinforced in one (1) dimension (e.g. length or width), reinforced in two (2) dimensions (e.g. both length and width), or reinforced in three (3) dimensions (i.e. length, width and depth). For example, an implantable contact lens (icl) or phakic refractive lens (prl) configured to fit between the natural lens and the iris, or between a previously implanted intraocular lens (IOL) and the iris. in the posterior chamber of the eye can be reinforced with a reinforcement(s) so as to maintain its shape during use in the eye. However, the implantable lens device is still configured to be implantable through a small incision in the eye with forceps or a lens injector. Specifically, a vaulted type phakic refractive lens can be provided with a reinforcement portion to maintain its shape when implanted in the space between the natural crystalline lens and iris to maintain its spacing and no contact with the natural crystalline lens so as to prevent cataracts even when the iris is pressed or presses against the vaulted phakic refractive lens. More specifically, the phakic refractive lens (prl) can be configured to bridge or span over a portion or the entire anterior surface of the natural crystalline lens (e.g. bridge over at least a center portion of the natural crystalline lens) while the lens portion of the phakic refractive lens resists deforning or changing shape to prevent contact with at least the center and preferably the entire natural crystalline lens, again to prevent cataracts. In a preferred embodiment of the phakic refractive lens, the lens optic portion of the phakic refractive lens is sufficiently reinforced to substantially prevent contact with the natural crystalline lens. Further, it is preferred that the lens optic device be reinforced so as to have shape memory in the event the lens optic portion, lens haptic portion (e.g. plate haptic portions) or the overall phakic refractive lens is bent, curved or otherwise deformed so as to prevent or minimize contact with the natural crystalline lens, and possible the back side of the iris.

The lens material used for the implantable lens devices according to the present invention is preferably a soft, resilient, bendable, foldable or otherwise deformable and biocompatible material such as silicon, collagen-containing polymer, acrylic polymers, and other know suitable biocompable implant materials. The reinforcement material preferably includes, but is not limited to polymer, synthetic polymer, plastic, plastic particles, plastic fibers, plastic strands, thermoplastic, polyamide, polyester, polyamide, glass, fiberglass, polymethyl methacrylate (PMMA), optic fiber, nylon, Kevlar, carbon, carbon fiber, ceramic, ceramic fiber, boron, boron fiber, metal, metal fiber, cobalt, cobalt fiber, composite, composite fiber in various forms including, but not limited to powders, particles, strands, filaments, mono-filaments, fibers, wires, cables, sheets, molded, molded shapes, extruded, extruded shapes, machined, machined shapes, laser shaped, vacuum formed, welded, thermo welded and thermo formed. The reinforcement portion can be made of biocompatible material, coated, layered, wrapped, or enveloped with a biocompatible material, microencapsulated, treated to be biocompatible, or embedded in the lens material so as to be biocompatible.

A preferred embodiment of the implantable lens device according to the present invention is a hingeless lens device. This preferred implantable lens device can be configured to be highly flexible and significantly bend along its length, or at one or more particular locations along its length and/or width. One particularly preferred embodiment, allows for significant being at the connection between the lens optic portion and lens haptic portion without the use of a hinge. For example, providing a reinforcement in the lens haptic portion and/or lens optic portion, but not at the connection therebetween, concentrates bending forces at the unreinforced connections causing significant bending without a hinge structure or arrangement. Another preferred embodiment includes hinged connections located between the lens optic portions and lens haptic portions or at other locations or multiple locations along the length of the lens haptic portion to enhance bending at these locations.

A preferred embodiment of the implantable lens device according to the present invention includes a reinforced or stiffened lens haptic portion, in particular a lens plate haptic porton. Specifically, a portion or portions of the lens haptic portion are reinforced from and outer edge to a point or location of connection with the lens optic portion. For example, an implantable lens device is configured with a lens optic portion and a pair of opposed lens plate haptic end portions. The lens plate hapic end portions extend from the point or location of connection with the lens optic portion to the outer ends thereof. The lens plate haptic end portions are reinforced to strengthen or stiffen the lens plate haptic portion along at least the length thereof. This reinforced or stiffened arrangement is effective to transfer forces (e.g. compression, tension, and/or shear forces) exterted on the edges and surfaces of the lens plate haptic end portions by the eye tissue (i.e. capsular bag, zonules and surrounding accommodative eye musculature) to the point or location of the connection with the lens optic portion. In this manner, the forces exterted on the lens plate haptic end portions is transmitted and concentrated at these connections to enhance bending at these connections. Specifically, many of the lens materials(e.g. silicone, acrylic polymer, collagen-containing polymer) unreinforced are unable to carry or transmit forces due to their low material strength (e.g. low compressive strength, low to medium tensile strength, low shear strength), and are structurally highly pliable. These lens materials are desireable to allow folding, bending, rolling or compression of the lens device to allow insertion through a small incision in the eye (e.g. 2.5 millimeters or smaller). The present invention utilizes the combination of these types of lens materials in combination with a reinforcement or reinforcements to strengthen the implantable lens device in at least one dimension, in particular along a portion or portions of the length of the lens plated haptic portion while allowing the implantable lens device to still be folded, bent (e.g. along longitudinal axis), rolled or compressed (e.g. along width) to still allow insertion through a small incision in the eye.

A preferred embodiment of the implantable lens device according to the present invention includes a reinforced or stiffened or stabilized lens optic portion. It is important that the lens optic portion remain optically functioning and optically stable during operation requiring structurally stability of the lens optic portion, especially when the implantable lens device is an accommodating lens device having a moving lens optic portion. The lens optic portion is provided with one or more reinforcements to strengthen or stiffen the lens optic portion. For example, a ring-shaped reinforcement is provided in the lens optic portion outside the center optically functioning zone of the lens optic portion. This reinforcement strengthens and stabilizes the lens optic portion, while still allowing for folding, bending, rolling, or compression of the lens optic portion to still allow insertion of the implantable lens device through a small incision in the eye.

### BRIEF DESCRIPTION OF THE DRAWINGS

FIG. 1 is a perspective view of a first preferred embodiment of a lens device according to the present invention.

FIG. 2 is a side elevational view of the lens device shown in FIG. 1 in a resting or unstressed condition.

FIG. 3 is a side elevational view of the lens device shown in FIG. 1, in a stressed condition under tension.

FIG. 4 is a top planar view of the lens device shown in FIGS. 1, 2, and 3.

FIG. 5 is a side elevational view of a second preferred embodiment of a lens device according to the present invention in a resting or unstressed condition.

FIG. 6 is a side elevational view of the intraocular lens device shown in FIG. 5, in a stressed condition under tension.

FIG. 7 is a top planar view of a third preferred embodiment of a lens device according to the present invention.

FIG. 8 is a cross-sectional view of the lens device, as indicated in FIG. 7.

FIG. 9 is a cross-sectional view of the lens device, as indicated in FIG. 7.

FIG. 10 is a side elevational view view of the lens device shown in FIG. 7, in a resting or unstressed condition.

FIG. 11 is a sice elevational view view of a lens device shown in FIG. 7, is a stress condition under tension.

FIG. 12 is top planer view of a fourth preferred embodiment of a lens device according to the present invention.

FIG. 13 is a side elevational view of the device shown in FIG. 12.

FIG. 14 is a top planar view view of a fifth preferred embodiment of a lens device according to the present invention.

FIG. 15 is a side elevational view of the lens device shown in FIG. 13, in an unstressed resting condition.

FIG. 16 is a cross-sectional view of the lens device, as indicated in FIG. 14.

FIG. 17 is a top planar view of a sixth preferred embodiment of a lens device according to the present invention.

FIG. 18 is a side elevational view of the lens device, as indicated in FIG. 17.

FIG. 19 is a cross-sectional view of the lens device, as indicated in FIG. 17.

FIG. 20 is a partial broken away perspective view of the matrix lens insert or reinforcement of the lens device shown in FIG. 17.

FIG. 21 is a top planar view of an seventh preferred embodiment of a lens device according to the present invention.

FIG. 22 is a cross-sectional view of the lens device, as indicated in FIG. 21.

FIG. 23 is a partial broken away side elevational view of the leg portion of the lens device shown in FIG. 1.

FIG. 24 is a partial broken away side elevational view of the leg portion of the lens device shown in FIG. 5.

FIG. 25 is a partial broken away side elevational view of a modified leg portion of a lens device according to the present invention.

FIG. 26 is a partial broken away top planar elevational view of the leg portion of a lens device according to the present invention.

FIG. 27 is a partial broken away side elevational view of the leg portion of a lens device according to the present invention.

FIG. 28 is a side elevational view of the lens device shown in FIG. 1, in a stressed condition when compression forces are applied to opposite ends of the lens haptic portion.

FIG. 29 is a side elevational view of the lens device shown in FIG. 1. in an unstressed or resting position (i.e. no tension or compression or shear forces applied to opposite ends of the lens haptic portions).

FIG. 30 is a side elevational view of the lens device shown in FIG. 1, in a stress condition when tension forces are applied to opposite ends of the lens haptic portions.

FIG. 31 is a top planar view of an eighth preferred embodiment of lens device according to the present invention.

FIG. 32 is a side elevational view of the lens device shown in FIG. 31.

FIG. 33 is a top planar view of a ninth preferred embodiment of the lens device according to the present invention.

FIG. 34 is a side elevational view of the implantable lens device shown in FIG. 33, when in an unstressed or resting position.

FIG. 35 is a side elevational view of the implanatable lens device shown in FIG. 33, when compression forces are applied to opposite ends of the lens haptic portion.

FIG. 36 is top planar view of a tenth preferred embodiment of the lens device according to the present invention.

FIG. 37 is a side elevational view of the lens device shown in FIG. 36.

FIG. 38 is top planar view of an eleventh preferred embodiment of the lens device according to the present invention.

FIG. 39 is a top planar view of a twelfth preferred embodiment of the lens device according to the present invention.

FIG. 40 is a side elevational view of the lens device shown in FIG. 39.

FIG. 41 is a side elevational view of an modified version of the lens device shown in FIG. 39.

FIG. 42 is a side elevational view of a further modified version of the lens device shown in FIG. 39.

FIG. 43 is a top planar view of a thirtenth preferred embodiment of the lens device according to the present invention.

FIG. 44 is a side elevational view of the lens device shown in FIG. 43.

FIG. 45 is a top planar view of a fourteenth preferred embodiment of the lens device according to the present invention.

FIG. 46 is a side elevational view of the lens device shown in FIG. 45.

FIG. 47 is a fifteenth preferred embodiment of the lens device according to the present invention.

FIG. 48 is a cross-sectional view of the lens device, as indicated in FIG. 47.

FIG. 49 is a sixteenth preferred embodiment of the lens device according to the present invention.

FIG. 50 is a side elevational view of the lens device shown in FIG. 49, in an unstressed or resting condition.

FIG. 51 is a side elevational view of the lens device shown in FIG. 49, in a stressed condition under compression.

FIG. 52 is a top planar view of a modified version of the embodiment of the lens device shown in FIG. 49.

FIG. 53 is a top planar view of another modified version of the embodiment of the lens device shown in FIG. 49.

FIG. 54 is a top planar view of a further modified version of the lens device shown in FIG. 49.

FIG. 55 is a top planar view of another further modified version of the lens device shown in FIG. 49.

FIG. 56 is a top planar view of an even further modified version of the lens device shown in FIG. 49.

FIG. 57 is a top planar view of even another modified version of the lens device shown in FIG. 49.

FIG. 58 is a top planar view of a seventeeth preferred embodiment of the lens device according to the present invention.

FIG. 59 is a top planar view of an eighteeth preferred embodiment of the lens device according to the present invention.

FIG. 60 is a top planar view of a nineteeth preferred embodiment of the lens device according to the present invention.

FIG. 61 is a cross-sectional view of the lens device, as indicated in FIG. 60.

### DETAILED DESCRIPTION OF PREFERRED EMBODIMENTS

An implantable lens device 10, in particular an accommodating lens device, is shown in FIGS. 1 to 4.

The implantable lens device 10 includes a lens portion 12 and plate haptic portions 14, 14. The lens portion 12 is connected to the plate haptic portion 14, 14 by leg portions 16, 16 located on opposite ends of the lens portion 12.

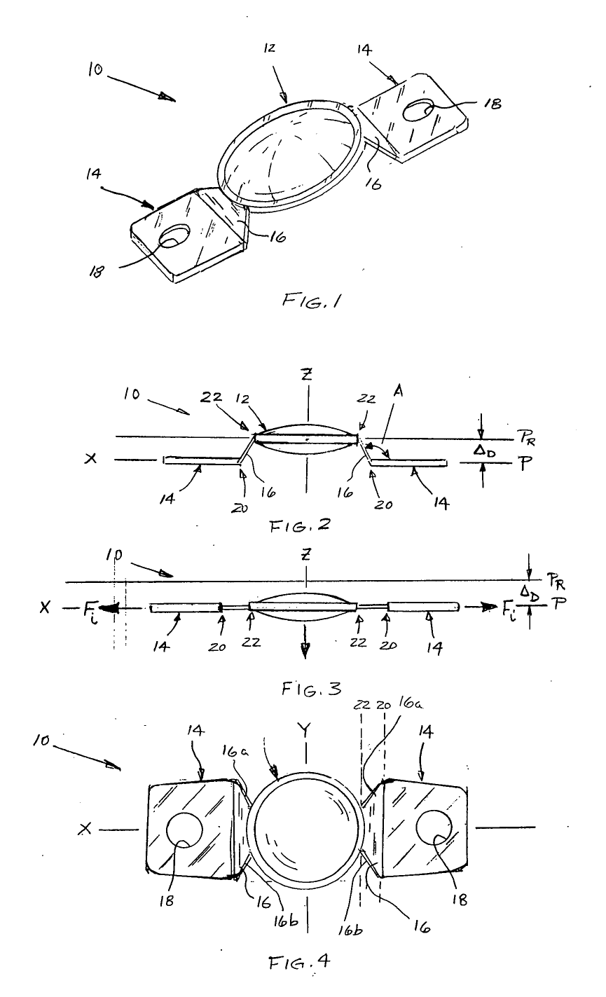

As shown in FIG. 2, the leg portions 16, 16 are bent relative to the lens portion 12 and the plate haptic portion 14, 14 when the intraocular lens device 10 is in a resting state or unstressed condition. When tensile force Fᵢ is applied to opposite ends of the plate haptic portions 14, 14, the bent leg portions 16, 16 unbend and straighten out until aligned with the direction of the tensile force Fᵢ, as shown in FIG. 3. In this manner, the lens portion 12 traverses a distance Δ_{D1} to provide accommodation of the intraocular lens device 10. When a compression force is applied to opposite ends of the plate haptic portions 14, 14, the bent leg portions 16, 16 bend more, and the lens portion 12 traversed an opposite distance in the opposite direction to provide accommodation of the intraocular lens device 10.

As shown in FIG. 4, the leg portions 16, 16 taper inwardly from the width of the plate haptic portions 14 to connection with the lens portion 12. The leg portions 16, 16 have inwardly tapering edges 16a and 16b. Preferably, the thickness of the leg portions 16, 16 is less than the plate haptic portions 14, 14 to facilitate unbending and straightening of the leg portions 16, 16 with the plate haptic portions 14, 14.

The leg portions 16, 16 unbend along hinge axis 20, 20 and hinge 22, 22 as shown in FIG. 4.

The leg portions 16 are set at an angle A relative to the plate haptic portions 14, as shown in FIG. 2. The angle A is preferably 45° to a 135°. The angle A can be adjusted to adjust the distance Δ_{D} desired or prescribed for a particular patient.

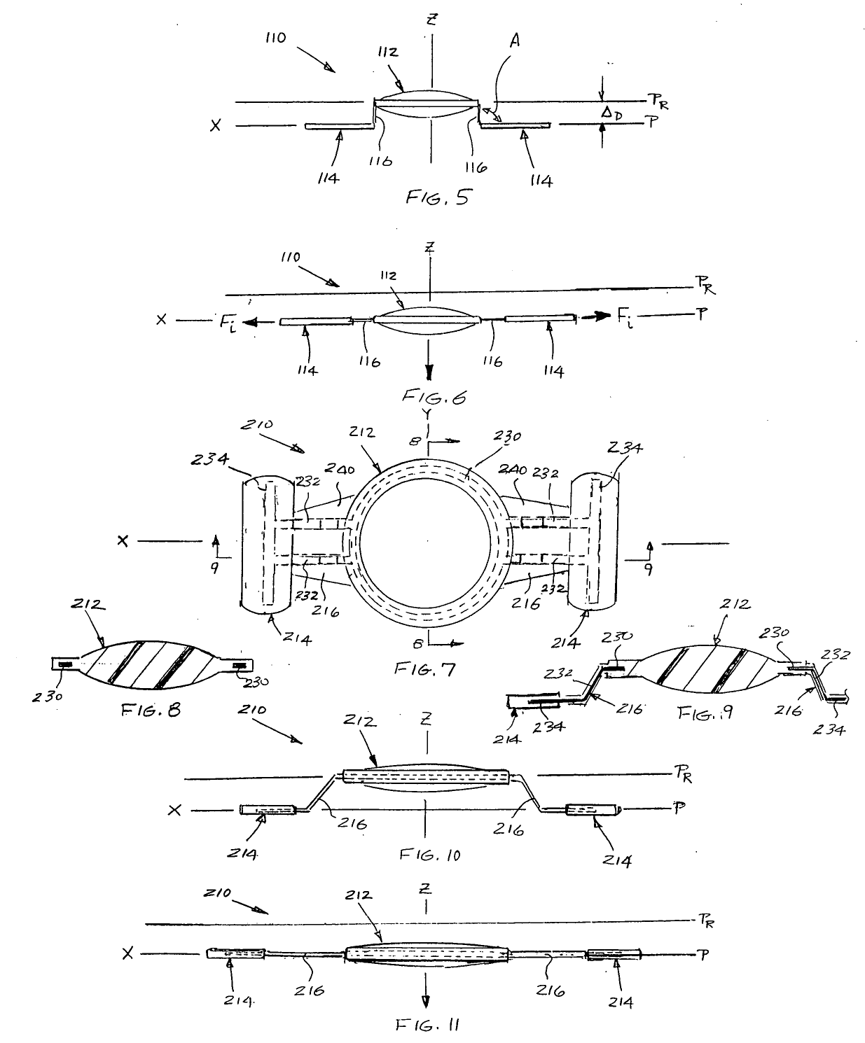

Another embodiment of the implantable lens device 110 according to the present invention is shown in FIGS. 5 and 6.

The implantable lens device 110 includes a lens portion 112 connected to a pair of plate haptic portions 114, 114 by leg portions 116, 116. In this embodiment, the angle A between the plate haptic portion 114 and the leg portion 116 is approximately ninety degrees (90°). This arrangement will provide the maximum throw distance Δ_{D} during operation thereof. The implantable lens device 110 is shown in the resting position, and when a tension force is applied to the outer edges or peripheral of the plate haptic portions 114, the leg portions 116 unbend relative to the lens portion 112 and the plate haptic portions 114 and straighten out to some extent or to a full extent, as shown in FIG. 6, depending on the amount of the tension force and the amount of accommodation required by the eye.

Another embodiment of the implantable lens device 210 according to the present invention is shown in FIGS. 7 to 9.

The implantable lens device 210 includes a lens portion 212 connected to a pair of plate haptic portions 214, 214 by arm portions 216, 216. The lens portion is provided with a ring-shaped insert 230, as shown in FIGS. 7 and 8. The insert 230 can be made from a variety of materials the same as different as the lens portion 212. The ring-shaped insert 230 can be configured to reinforce the lens portion 212. For example, the insert 30 can be made of a reinforcing material such as polyamide, polyester, polysulfone, acrylic polymer or other suitable material. The insert 30 is encapsulated within the resin material of the lens portion 212. Alternatively, the insert 30 can be adhered or weld to one side of the lens portion 212. In some embodiments, the insert 30 is unstressed during manufacturing of the intraocular lens device 210, or alternatively, the ring-shaped insert is pre-stressed (e.g. compressive force applied to ring, tension force applied to ring) (e.g. uniform tension force applied around ring, uniform compression force applied around ring, local tension force applied to portion of ring, local compression force applied to ring, uniform tortional force applied to ring, local tortional force applied to portion of ring, or combination thereof). The insert 30 can be configured to reinforce the lens portion 212 against bending or otherwise to stabilize the lens portion 212 to provide optical stability.

The leg portions 216 are provided with an insert(s) configured to reinforce the leg portions along the X axis (i.e. unidirectional) against compression and/or tension depending on the lens design. The inserts 232 can be unstressed during manufacture of the intraocular lens device 210, or can be placed under tension, compression and/or tortional stresses along the lengths thereof or at portions or points along the length thereof. The plate haptic portions 214 are also provided with inserts 234, 234, which reinforce the plate haptic portion 214 in the Y axis direction (i.e. unidirectional). The inserts 234 can be unstressed during manufacture of the intraocular lens device 210, or alternatively, can be pre-stressed by tension, compression and/or tortional stresses prior to or during manufacturing of the intraocular lens device 210. Again, the pre-stressing forces can be applied uniformly, regionally, locally and/or point forces. The entire insert or portions of the insert can be pre-shaped (e.g. molded, bent, configured) to have a particular shape prior to molding the lens material around the insert. Preferably, the insert is a reinforcement insert configured, designed or specified to increase the strength, shape stability, optical stability, movement stability and enhanced accommodation of the implantable lens 210 during use in the eye.

The inserts 230, 232 and 234 can be separate pieces or components, or can be made as a single unit. For example, when molding a deformable silicon intraocular lens, the insert 230, 232 and 234 are made from a single sheet cut out of polyamide or polyester, the one-piece insert is then placed in the mold cavity, not-stressed or pre-stressed, for example by pulling, pushing, twisting ends or edges of the one-piece insert, and then filling the mold cavity with a resin which encapsulates the one-piece insert during the molding process, and then heating the mold to polymerize and cross-link the molding resin. In this manner, a one-piece intraocular lens device 210 is made.

As an optional feature, a very thin section of resin is molded around the inserts 232 of the leg portions 216, 216 to provide webbing 240 to encapsulate the inserts 32 to protect the inserts 32 and/or cover the inserts 32 so as not to damage tissues in the eye once implanted. For example, the thickness of the resin of the webbing 40 can be the same thickness as the plate haptic portions 214, but are preferably are of less thickness then the plate haptic portions 214, 214. In some embodiments, the thickness of the resin of the webbing 240 is less thick than the inserts 232, and thus the inserts 232 with a resin layer thereon protrude somewhat from the rest of the surface or plane of the webbing 240 providing bumps or protrusions in the surface of the leg portions 216 where the inserts 232 are located.

The inserts 230, 232 and 234 again can be made from a wide variety of materials, including but not limited to polymer, plastic, thermo plastic, silicon, acrylic polymer, polymethyl methacrylate (PMMA), polyamide, polyester, polycarbonate, fiberglass, Kevlar, graphite, ceramic, glass, metal, metal composite, polymer composite or other suitable material. The material can be uniform throughout its dimensions, or can be fabricated to vary linearly, exponentially, continuously, discontinuously along the three-dimensional axis of the insert. For example, the insert can be configured, designed, fabricated or otherwise made to tailor to vary in thickness, strength, shape, chemical composition, tensile strength, compressive strength, tortional strength, hardness, surface finish, surface texturing, or other engineering parameters or variables typical of materials in one or more directions along the three-dimensional axis of the insert. The insert, for example, can have a variety of different transverse cross-sectional shapes such as circle, triangle, square, rectangular, multi-sided (e.g. hexagonal), symmetrical, asymnetrical, tubing, star-shaped, serrated edges, u-shaped, l-shaped, etc. The insert can be a composite device such as a multi-layered implant, reinforced in one or more directions with inserts within the inserts, varying layers of polymerization along the lengths and/or thickness thereof. The surface of the inserts can be treated (e.g. radiated, chemically etched, heated, annealed, sanded, shot penned, glass beaded, roughened, machined) to facilitate adhesion and/or connection with the surrounding resin material embedding the insert. Alternatively, the surface of portions of the surface of the insert can be configured or treated so that the insert does not adhere to the embedding resin material so that the insert slides or slips within the lens material, even after polymerizing or cross-linking the embedding resin material.

The inserts can be pre-stressed prior to placing in a mold cavity and/or in the mold cavity by applying tensile force, compressive force and/or tortional force at one or more positions, areas or volumes of the insert material. Alternatively, or in addition, the insert can be pre-stressed by cooling, heating, steaming, radiating, curing, polymerizing, further polymerizing, or by other known methods or techniques, prior to insertion in the mold, in the mold itself and/or after formation or manufacturing of the intraocular lens device. For example, the implantable lens device is manufactured without pre-stressing the insert, however, the insert is treated with electromagnetic waves (e.g. laser) while being embedding in the polymer resin material prior to implantation in the eye. Of course, this intraocular lens device, in particular the insert of the intraocular lens device, can be further stressed or unstressed using electromagnetic radiation in vivo, after implantation within the eye.

Another embodiment of the intraocular lens device 310 according to the present invention is shown in FIGS. 12 and 13.

The implantable lens device 310 includes a lens portion 312 connected to a pair of plate haptic portions, 314, 314 by leg portions 316, 316. The lens portion 312 includes a circular-shaped insert 330a at or near the perimeter of the lens portion 312, and optionally an inner circular-shaped insert 330b. The insert 330a has a rectangular or flat cross-sectional shape, as shown in FIG. 13 and the inner circular-shaped insert 330b has a circular cross-sectional shape shown in FIG. 13. The inserts 330a and 330b can be made from optically or substantially optically clear material (e.g. polyprolene) or can be made from partially or non-optically clear material, however, dimensioned so as to not optically interfere in a substantial or meaningful manner with light rays passing through the lens portion 312. Thus, the inserts shown in FIGS. 12 and 13 may be significantly exaggerated in size versus the actual size of the implants for illustration purposes.

In this embodiment, the leg portions 316, 316 are not provided with inserts to facilitate flexibility or bending thereof. The inserts 330a and 330b can be unstressed or pre-stressed in the manufactured implantable lens device 310 prior to implantation in the eye. Once implanted in the eye, in vivo, the implants 330a and 330b can be treated with electromagnetic radiation uniformly, along portions thereof (e.g. at point, axis, volume positions) to change the refractive characteristics of the lens portion (e.g. change surface properties of lens portions 312 and/or change the radius of curvature plus or minus at or near the locations of the implants). The plate haptic portions 314 are provided with the implants 334, and configured in a manner to reinforce the plate haptic portions 314 in two (2) dimensions along the plane containing the plate haptic portions 314. Thus, the compressive and/or tensile strength between the opposite ends along the length of the plate haptic portions 314 are increased by the inserts 334 to facilitate accommodating compression and/or tension forces applied along the length (axis X of the intraocular lens device 310).

The plate haptic portions 314 are provided with through holes 318 to facilitate anchoring the plate haptic portions within the eye, in particular when implanted in the capsular bag of the posterior chamber of the eye. Alternatively, or in addition, the implantable lens device 310 can be a phakic refractive lens (prl) type implantable lens device to be located in the posterior chamber of the eye between the natural crystalline lens or prior implanted intraocular lens and the iris.

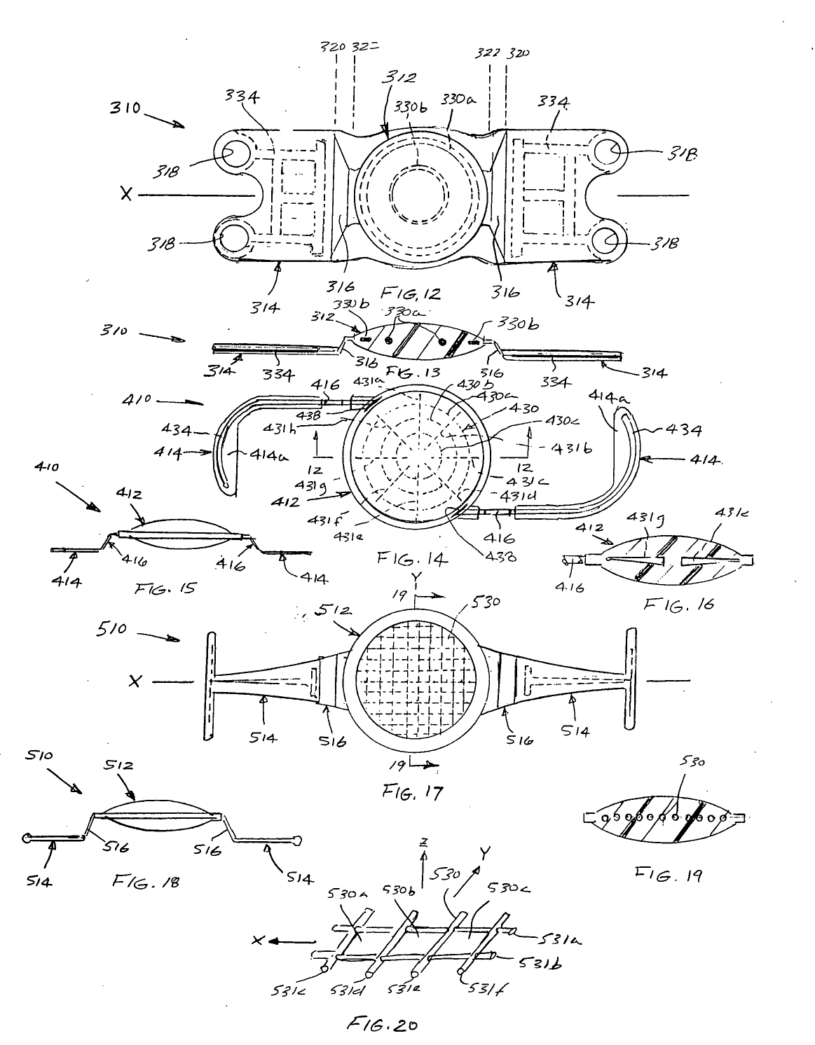

Another embodiment of the intraocular lens device 410 according to the present invention is shown in FIGS. 14 to 16.

The intraocular lens device 410 is configured somewhat as a loop-type intraocular lens device. The intraocular lens device 410 includes a lens portion 412 connected to a pair of loop-type haptic portions 414, 414 by a pair of leg portions 416, 416.

The lens portion 412, 412 are provided with an insert that can be multiple pieces or a single piece. The insert 430 includes circular-shaped insert portions 430a and 430b and 430c and straight radial oriented inserts 431a-h. As shown in FIG. 16, the straight radial oriented inserts 431c and 431g are shown as being tapering from a wider thickness towards the center of the lens portion 412 to a thinner thickness towards the outer perimeter of the lens portion 412. This arrangement is to allow more thickening of the center portion of the lens portion when electromagnetic radiation, such as laser light, is applied to the more center thicker portions of the inserts 431c and 431g.

The loop haptic portions 414 included a polymer resin layer 414a provided with inserts 434 configured to reinforce and strengthen the loop haptic portions 414, 414. The leg portions 416 are extensions of the inserts 434 and extend to the lens portion 412, and anchored by anchor portions 438 in the perimeter of the lens portion 412.

Another embodiment of the intraocular lens device 510 according to the present invention is shown in FIGS. 17 to 20.

The intraocular lens device 510 includes a lens portion 512 connected to a pair of haptic portion 514, 514 by a pair of leg portions 516, 516. The lens portion 512 is provided with an insert 530 configured as a matrix or mesh, preferably a fine matrix or mesh. The insert 530 can be separate pieces or a single piece. The matrix insert 530 defines a matrix of rectangles, preferably squares. However, the matrix can be configured to provide other pluralities of shapes such as triangle circle, multi-sided shapes, or a progressive matrix varying in the size of the openings in one (1), two (2), or possibly three (3) dimensions. The matrix insert 530 is configured to facilitate precise treatment by electromagnetic radiation, in vivo, after implantation of the intraocular lens device 510 within the eye. The matrix facilitates locating specific points within the lens portion 512, and a particular matrix can be treated with electromagnetic radiation to induce stress or relaxation, or further polymerization or unraveling of polymer strands, at a particular point or region of the lens portion 512. For example, a portion of the matrix implant is hit with laser light to condense a particular cell or groups of cells of the matrix implant and/or the polymer resin material within a cell is treated with laser light to cause compression and expansion of the dimensions of a particular cell or groups of cells. In this manner, the thickness, curvature, stiffness, strength, refractive light properties can be modified or varied in vivo, by treatment with electromagnetic radiation.

The matrix implant 530 can be a screen, woven mesh, threads, wires, strands of material, or can be a single unwoven matrix (e.g. screen, one piece made by injection molding). Again the matrix implant 530 is preferably made of optically clear material, or can be made from non-optically clear material, however dimensioned, sized or treated (e.g. electromagnetic radiation) so as to purposely interfere or purposely (i.e. designed) not to interfere with the light refractive properties of the lens portion 512.

As shown in FIG. 20, the matrix insert defines a plurality of cells 530a, 530b and 530c between horizontal strands 531a and 531b and vertical strands 531d-h. The matrix implant 530 is shown as being a non-woven mesh or screen configured in a single plane or configured to be spherically (i.e. curved along the X axis and Y axis in the Z direction).

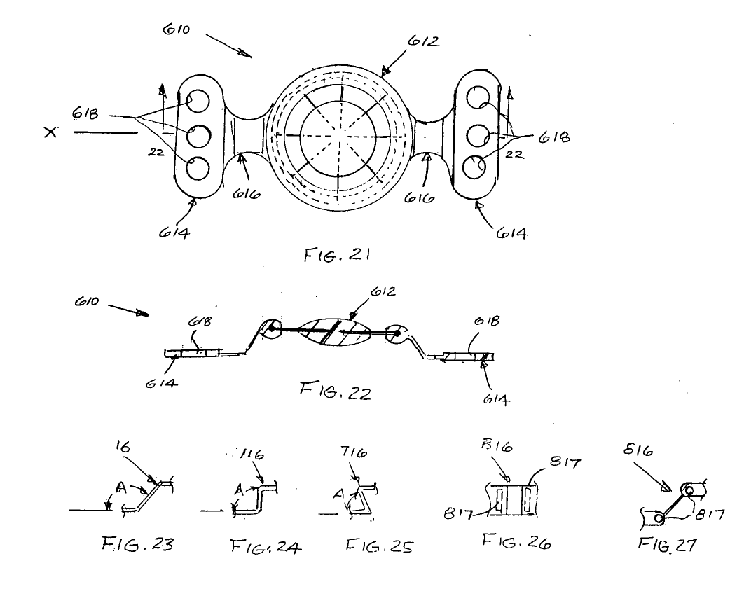

Another embodiment of the intraocular lens device 610 according to the present invention is shown in FIGS. 21 and 22.

The intraocular lens device 610 includes a lens portion 612 connected to a pair of plate haptic portions 614 by a pair of leg portions 616. The lens portion includes an optical lens portion 612a connected to an outer lens ring 612b by a plurality of spoke members 640. The spoke members can be extensions of inserts into the optical lens portion 612a and the outer lens ring portion 612b. The orientation, location, size, shape, configuration, refractive power, aspheric, spheric, power and other parameters and characteristics of the optical lens portion can be changed or addressed by use of electromagnetic radiation, in vivo, after implantation of the intraocular lens device 610 into the eye. Specifically, the electromagnetic radiation can be focused or directed to specific spoke members to the same or varying extent to create change (e.g. provide tension along the spoke members or compression of the spoke members).

A preferred embodiment of the intraocular lens device according to the present invention is an accommodating intraocular device. Specifically, the intraocular lens device is configured so that the lens portion thereof moves to some extent during operation of the eye to simulate the movement of the natural crystalline lens of the eye along the focal axis thereof prior to cataract removal. The accommodation function of the eye provides for a dioper change of the lens to facilitate a person's ability to read or look at object closely. The accommodating type intraocular lens device according to the present invention is to be used to restore accommodation for persons after a clear natural lens removal or a cataract lens removal, wherein the accommodating intraocular lens device is implanted into the capsular bag of the posterior chamber of the eye.

The intraocular lens device according to the present invention includes a lens portion connected to a haptic portion or pair haptic portions by one or more leg portions. The leg portions are preferably bent leg portions having a “bent” configuration when in a resting position. For example, the configuration of the intraocular lens device is shown in FIGS. 2, 5, 10, 13, 14, 18 and 22 show various embodiments of the intraocular lens device in resting positions (i.e. no tension forces being applied to the ends of the haptic portions) with the leg portions being in the “bent” configuration. The intraocular lens device according to the present invention is configured so that the bent leg portions unbend and straighten when tension forces are applied to the ends of the haptic portion or portions causing the lens portion to move towards a plane containing the haptic portions. When the tension force is released, the leg portions are relaxed and go from a more straightened position to the original bent position.

Various bent leg portions are shown in FIGS. 23 to 27.

The bent leg portions vary in angle relative to the lens portions and haptic portions of the intraocular lens device. The perpendicular embodiment shown in FIG. 224 provides maximum throw or movement of the lens portion relative to the haptic portion or portions. The angle A preferably ranges from 45 degrees to 135 degrees.

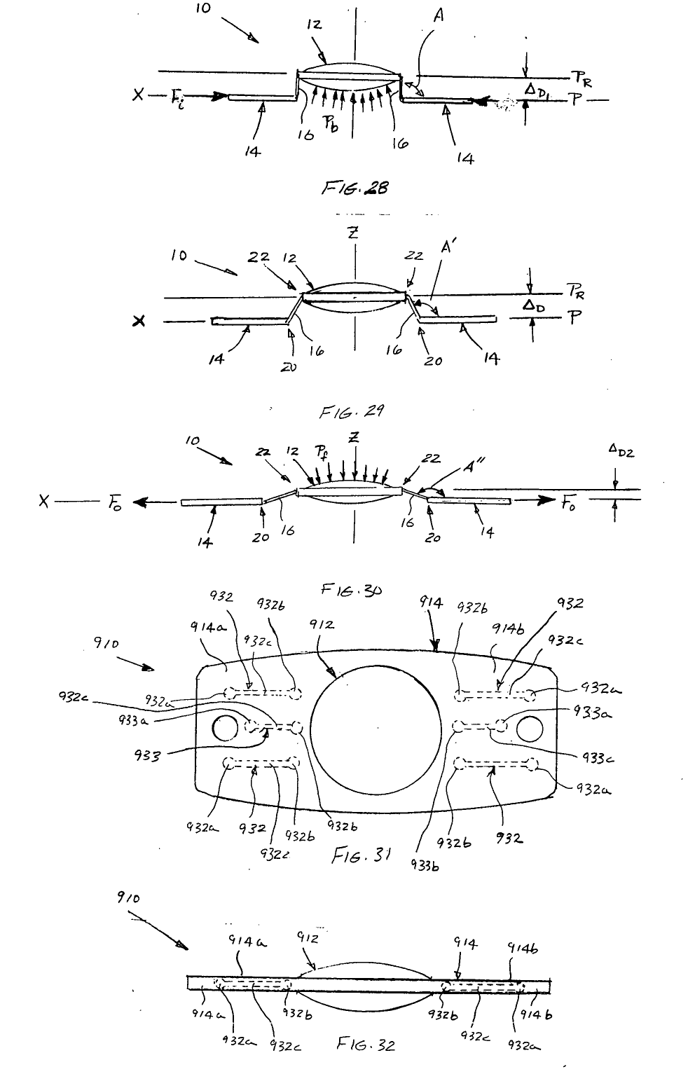

As shown in FIGS. 26 and 27, the leg portion 816 can be provided with rod-shaped implants 817, 817, configured to slid or slip within the thickness of the leg portions to enhance or facilitate bending along the bending axes thereof. The rod-shaped implants 817, 817 can be separate parts and operate independently, or can be linked or connected through the length of the leg portion 816 to reinforce the leg portion and still facilitate bending.

The intraocular lens according to the present invention can be implanted in the resting position (e.g. FIGS. 2, 5, 10, 13, 15, 18 and 22) in the capsular bag of the eye, and then connected (e.g. by sutures, fibrosis or healing connection) in place within the capsular bag of the eye in the resting position. Alternatively, the intraocular lens is stressed or placed in a prestressed condition so as to be under tension (lengthened) and/or under compression (shortened) and/or under shear (twisting), for example, by use of sutures, by use of a removable mechanical device for providing temporary tension or compression on the accommodating lens device, by subsequent manipulated in vivo, or by subsequent treatment with electromagnetic radiation, after being implanted in the eye, during the capsular bag healing process, or after the capsular bag healing process to acheive various levels or degrees of stress and shear forces acting back and forth between the accommodating lens device and surrounding eye tissue (e.g. capsular bag, zonules, eye muscles). The particular design or construction of the accommodating lens device may allow portions of the lens to be under tension, under compression and/or shear when prestressed in certain manners or modes. Alternatively, an unstressed accommodating intraocular lens device according to the present invention can be placed under stress after being implanted, healed and affixed within the capsular bag by applying electromagnetic radiation directly or indirectly to the implanted lens device to cause stressing (e.g. application of lazer light expands or compresses points, matrix, lines, surfaces, planes, nodes, volumes of lens material or reinforcement portion to cause micro or macro bending, torsion, tension, compression of lens optic portion, haptic portion(s) to change portion or overall size, shape, dimesions of the implant lens device so as to exert forces (e.g. stress and/or shear forces) from the implanted lens device onto the surrounding eye tissue, including the capsular bag, zonules, and surrounding accommodating eye muscularture.

The prestressing or subsequently stressing of the lens device allows for the accommodating intraocular lens device to exert forces (e.g. tension, compression and/or shear) on the surrounding eye tissue after implantation, healing, and affixation within the capsular bag. The subsequent selective release of the prestressing of the accommodating lens device in the healed eye after significantly affixation of the haptic portions to the eye tissue (e.g. between the outer edges of the walls of the capsular bag) will exert forces from the accommodating lens device to the eye tissue in a manner the same as or similar to a healthy fully accommodating eye so as to mimic the accommodating mechanism of the fully functioning accommodating eye containing a natural crystalline lens. Even more specifically, the accommodating mechanism of the eye must be significantly compromised or disabled causing a signifcant deminished accommodating capacity of the eye resulting from the lens removal operation (e.g. capsularexhis, phacoemulsification and removal of the natural crystalline lens), perhaps due to the structural loss of the removed anterior portion of the capsular bag and structural loss of the removed natural crystalline lens, now gone and unable to sustain or mantain tensile, compression and/or shear forces onto the zonules and surrounding accommodating eye musculature. Clearly, the structural integrity of the capsular bag and accommodating functioning thereof are lost by the removal of this significant eye tissue. The prestressed accommodating lens device according to the present invention implanted in a particular manner (e.g. prestressing of implanted accommodating lens device is released after sufficient healing and affixation of the lens device within the capsular bag to allow lens device to exert forces or remaining capuslar bag tissue, zonules and surround accommodating eye muscles) to perhaps restore the lost structural integrity and lost accommodating functioning of the eye by replacing or simulating or mimicing the stresses the natural crystalline lens and intact capsular bag exert on the zonules and surrounding accommodating eye tissue. As a example, the prestressed or subsequently stressed accommodating intraocular lens according to the present invention is configured and implanted in a manner so that the accommodating intraocular lens in under tension only after implantation in the healed eye functioning at different levels or degrees of tension during accommodating of the eye. As another example, the prestressed or subsequently stressed accommodating intraocular lens according to the present invention is configured and implanted in a manner so that the accommodating intraocular lens in under compression only after implantation in the healed eye functioning at different levels or degrees of compression during accommodating of the eye. As a further example, the accommodating intraocular lens according to the present invention is configured and implanted in a manner so that the accommodating intraocular lens in under tension or compression, at different times or simultaneously, after implantation in the healed eye functioning back and forth between tension or compression depending on the fluctuating overall size, in particularly the length, and degree of bending of the implanted accommodating intraocular lens device. More specifically, in certain applications the medium stress level throughout the lens material passes back and forth through tension and compression during the accommodative process of the eye with the zonules and surrounding muscular applying tension and then compression on the accommodating intraocular lens implanted in the capsular bag. As a simple model, tensile force or compression forces are applied by the eye tissue to the opposite edges of the accommodating intraocular lens accross the length dimensions of the accommodating intraocular lens in combination with a flucuating pressure differential on opposite sides of the capsular bag and lens device unit combined causing accommodative movement.

The possible modes of operation of the implantable lens device 10 are illustrated in FIGS. 28-30.

The configuration of the implantable lens device 10 in an unstressed or resting condition is shown in FIG. 29. The implantable lens device 10 is shown in FIG. 29 without any forces and/or pressure exerted thereon or otherwise in a neutral state. When inwardly directed forces Fᵢ are exerted on the ends of the lens haptic portions 14 and/or a differential pressure illustrated as Pb is exerted on opposited sides of the lens optic-portion 12, as shown in FIG. 28, the lens optic portion 12 moves upwardly (forward or backward within the eye depending on the lens orientation within eye) relative the neutral position shown in FIG. 29. When inwardly directed force Fᵢ are applied to the haptic portions 14 and/or pressure Pf is applied to the upper side of the lens portion 12 as shown in FIG. 30, the lens optic portion 12 moves downwardly (backward or forward depending on the lens orientation in the eye). In this arrangement, the implantable lens device 10 undergoes both compression and tension during accommodative movement. Alternatively, the implantable lens device can be configured and implanted so as to operate only under the tension mode (i.e. configurations between FIG. 29 and FIG. 30), or can be configured and implanted so as to operate only under compression mode (i.e. configurations between FIG. 29 and FIG. 28). It is noteworthy that the travel length or throw of the lens optic portion 12 is reduced by approximately one-half when operating solely in the tension only or compression only modes verses the combined tension/compression modes with this particular arrangement or design of the implantable lens device 10.

The implantable lens device 10 can be configured to allow the lens portion 12 to move or accommodate mainly by the pressure differentials illustrated by P_{b} or P_{f} exerted on the lens portion 12 depending on the design of the implantable lens device 10 (e.g. by providing shorter overall length of lens device 10 and/or making more flexible connections between lens optic portion 12 and lens haptics 14, 14). Alternatively, the implantable lens device 10 can be configured to allow the lens portion 12 to move or accommodate mainly due to the forces Fᵢ or Fₒ applied to the edges of the haptic portions 14, 14 (e.g. by providing a longer overall length of the lens device 10 and/or making less flexible connections between lens optic portion 12 and lens haptics 14, 14). Again, it is preferred that combined edge forces and differential pressures are utilized to move or accommodate the lens portion 12 to increase the degree or extent of accommodative movement of the lens portion 12 within the eye. In advanced versions of the implantable lens device 10, the amount or degree of accommodation may be purposely prescribed and designed into the implantable lens device 10 to enhance or even possibly limit the forward and/or rearward movement of the lens portion 12 within the eye. Further, the implantable lens device 10 can also potentially be designed to provide a linear or non-linear (e.g. exponential function) between the amount of force and/or pressure exerted on the implantable lens device 10 and the degree, rate, or extent of movement or accommodation of the lens portion 12 within the eye.

As shown in FIGS. 28, 29 and 30, the angle between the lens haptic portion 14 and the lens leg portion 16 changes, respectively, from A to A′ to A″. The degree of bending of the angle A′ can be augmented by stiffening the lens haptic portions along the length thereof by providing stiffeners or a reinforcement portion (e.g. inserts) according to the present invention. Further, the lens leg portion 16 can be stiffened along the lengths thereof by also embedding a stiffener or reinforcement portion according to the present invention. This will allow force concentration at the point or axis of bending 20, 22. Specifically, the stiffeners or reinforcement portions concentrate the bending moments at the axes 20, 22 causing the main lens material to be subject to a higher level of bending moment at these axes. This allows for a greater degree or extend of accommodative movement of the lens portion 12.

The inwardly directed force Fᵢ (FIG. 28) and the outwardly directed forces Fₒ (FIG. 30) are shown diagrammatically. Specifically, these tension and compression forces represent forces applied to the edges of the lens haptic portions 14 combined with surface shear forces applied to the upper and lower surfaces of the lens plate haptic portions 14 by the eye tissue, mainly the capsular bag, zonules and surrounding accommodating eye muscularture. More specifically, after the implantable lens device 10 has been implanted, for example, in the capsular bag of the eye after a cataract lens removal, fibrosed tissue of the capsular bag adheres to the edges and surfaces of the lens plate haptic portions 14. The fibrosed tissue of the capsular bag is stressed inwardly or outwardly by the zonules and surrounding muscularture of the eye, which forces are transmitted from the tissue to the edges and surfaces of the lens plate haptic portions 14. Depending on the extent of adherence of the tissue to the surfaces of the lens plate haptic portions 14 dictates the extend or degree the forces are transmitted from the tissue to the implantable lens device 10. In addition, the forces directly applied to the edges of the lens plate haptic portions 14 by the tissue of the eye are effective with regards to inwardly directed compression forces and may be less effective with regards to outwardly directed tension forces due to the limited area of adherence of the fibrosed tissue on the edges of the lens plate haptic portions 14. Thus, the extend or degree of mechanical coupling or connection between the eye tissue and the lens plate haptic portions 14 can be designed or tailored depending on the performance and characteristics desired of the implantable lens device 10. Preferably, there exists a strong bond or mechanical connection between the eye tissue and the lens plate haptic portions 14 to maximize the degree or extent of accommodative movement of the lens portion 12. However, in some special applications, the lens plate haptic portions 14 are designed, configured, treated, coated or otherwise made so as to adhere or not adhere to the eye tissue to allow the lens plate haptic portions to float or move freely within the eye. For example, the edges and/or surfaces of the plate haptic portions can be frosted, mechanically etched, chemically etched, machined, designed, shaped, edged shaped, chemcially treated to enhance or decrease fibrosis or tissue bonding depending on the particular design of the lens device and application.

An eighth embodiment of the implantable lens device 910 is shown in FIGS. 31 and 32.

The implantable lens device 910 includes a lens optic portion 912 and a lens plate haptic portion 914 having opposed lens plate haptic end portions 914a, 914b. The lens plate haptic end portions 914a 914b are provided with reinforcements or reinforcement portions 932, 933. The reinforcements 932, 933 can be a structural stiffener or tensioner to increase the bending strength, tensile strength, and/or compression strength of the lens plate haptic end portions 914a, 914b. For example, the reinforcements 932, 933 can be made of fiber, composite fiber, fiberglass, carbon fiber, polyamide, polyimide, polysulfone, Proline or other suitable material to stiffen the lens plate haptic end portions 914a, 914b and/or increase the tensile strength thereof. In an embodiment in which the reinforcement portions 932, 933 are structural stiffeners, inwardly directed forces (inwardly directed compression forces) exerted by the eye tissue onto the edges and/or surfaces of the lens plate haptic end portions 914a, 914b transmit the force across the length of the lens plate haptic end portions 914a, 914b, and concentrate the force at the inward ends of the stiffeners located near the lens optic portion 912 to cause bending of the lens plate haptic end portions 914a, 914b at the connection or attachment point(s) to the lens optic portion 912. In this manner the implantable lens device 910 can be an accommodating lens or accommodating intraocular lens.

The ends 932a, 932b of the reinforcement 932 and the ends 933a, 933b of the reinforcement 933 can be provided with expanded head portions (two-dimensional or three-dimensional head portions) to spread the tension, compression and/or shear forces at the interface surface or connection between the head portions and the lens material of the lens plate haptic ends portions 914a, 914b so that the ends of the reinforcements 932, 933 do not puncture, tear, separate, cut, compromise or otherwise damage the lens material immediately surrounding the ends of the reinforcements 932, 933. The reinforcements 932, 933 can be surface treated and/or coated (e.g with a primer or adhesive) to ensure good to excellent surface bonding between the reinforcements 932, 933 and the surrounding lens material. Alternatively, portions of the reinforcements 932, 933 (e.g. center portions) can be treated or coated (e.g. with Teflon coating) so as to purposely not adhere to the surrounding lens material while the head portions of the reinforcements 932, 933 are purposely treated or coated to substantially adhere to the surrounding lens material to allow or provide slippage of the center portions 932c, 933c of the reinforcements 932, 933 to increase the degree or extent of transmittal of compression or tension forces along the lengths of the reinforcements 932, 933 while the ends 933a, 933b are thoroughly anchored to the lens material.

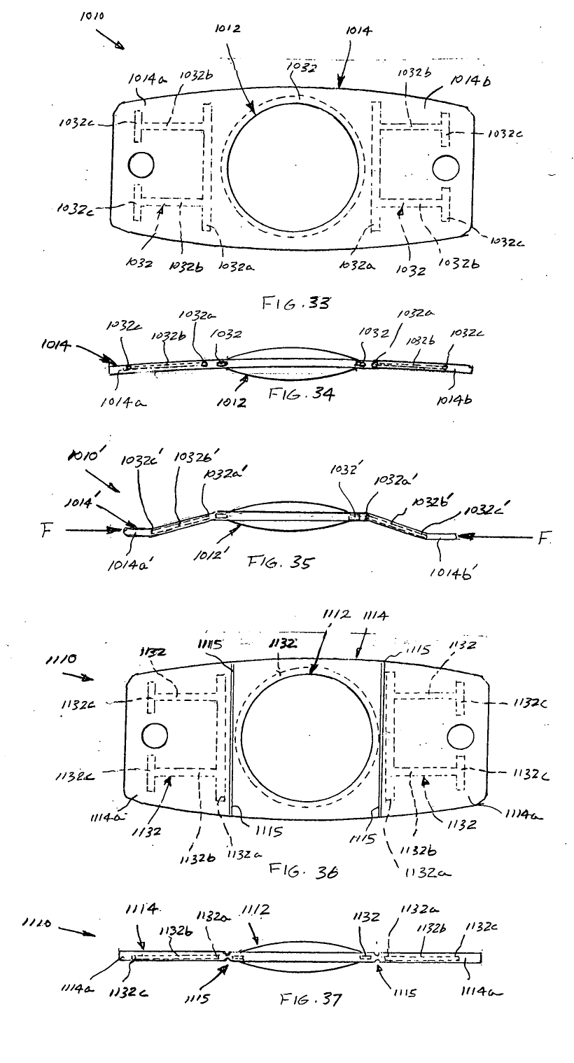

A ninth embodiment of an implantable lens device 1010 according to the present invention is shown in FIGS. 33 and 34.

The implantable lens device 1010 includes a lens optic portion 1012 and a lens plate haptic portion 1014. The lens plate haptic portion 1014 includes lens plate haptic end portions 1014a, 1014b.

The lens optic portion 1012 is reinforced and stabilized by a ring-shaped reinforcement 1032 surrounding, and concentric with the lens optic portion 1012. The ring-shaped reinforcement 1032 is embedded within of the lens plate haptic portion 1014, as shown in FIG. 34. Alternatively, the ring-shaped reinforcement 1032 can be layered, bonded, and/or mechanically connected to the surface of the lens portion 1012. The ring-shaped reinforcement 1032 can have a substantially flat configuration, round configuration or other specifically designed configuration (i.e. special outer perimeter shape and transverse cross-sectional shape such as triangular, square, polyhedron, star shaped). The ring-shaped reinforcement 1032 can increase the bending strength, tensile strength, and/or compressive strength around the permimter or edge of the lens optic portion 1012, for example, to prevent bending, distortion or decease performance of the lens optic portion 1012 during use, in particular during accommodative movement. Further, the ring-shaped reinforcement 1032 can have a substantial band strength to prevent distortion of the outer perimeter of the lens portion inwardly or outwardly, for example, from circular to oval shaped when inwardly directed compression forces or outwardly directed tension forces, and/or shear forces are exerted by eye tissue on the lens device, in particular on the edges of the lens plate haptic portion 1014.

The lens plate haptic end portions 1014a, 1014b are each provided with a reinforcement 1032 embedded within the thickness of the lens plate haptic end portions 1014a, 1014b. The reinforcement 1032 can be configured to increase the bending strength, tensile strength, and/or compressive strength of the lens plate haptic end portions 1014a, 1014b. The reinforcements 1032 include traversed members 1032a spaced apart and located adjacent to the ring-shaped reinforcement 1012. A pair of leg members 1032b extend from each traversed member 1032a, and include traversed end members 1032c. The reinforcement 1032 can have a flat profile (e.g. cut from a sheet of polyimide), can be rod-shaped (e.g. extruded), or can be custom shaped (e.g. molded). The lens plate haptic portion 1014′ includes lens plate haptic end portions 1014a′, 1014b′ set off at an angle relative to the remander of the lens plate haptic portion 1014′ when the lens device 1010 is place under compression. This configuration aligns the lens plate haptic end portions 1014a′, 1014b′ with the inwardly directed compressive force F and places the portion of lens plate haptic portion 1014 immediately surrounding the lens optic portion 1012′ in a spaced apart plane relative to the plane containing the lens plate haptic end portions 1014′a, 1014b′. This configuration ensures that the lens plate haptic portion 1014′ properly bends to raise and lower the lens optic portion 1012′ when compressive force F is applied to the ends of the lens plate haptic portion 1014′

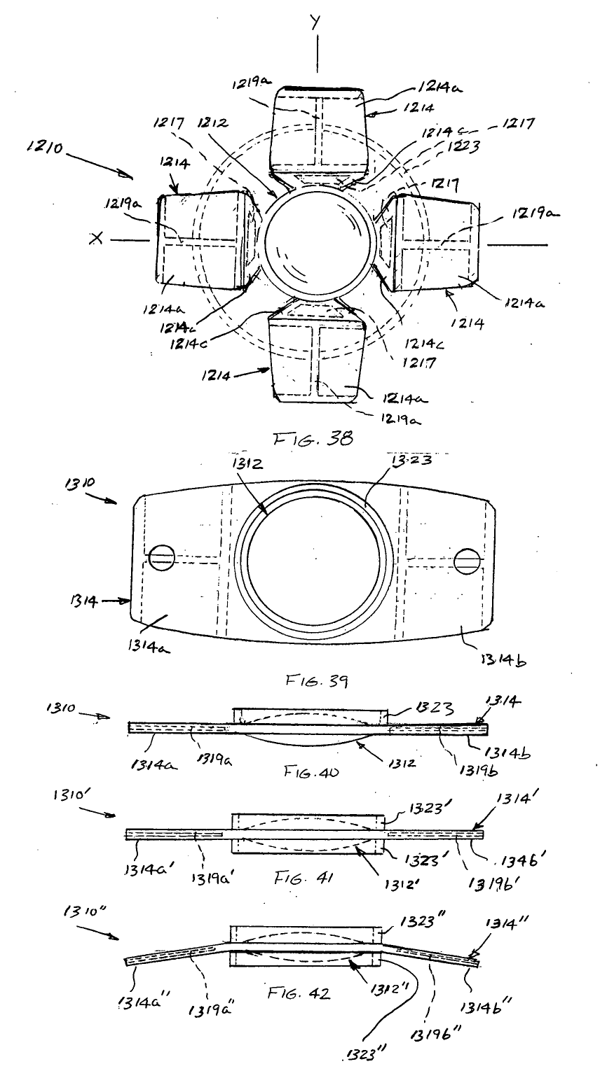

A tenth embodiment of an implantable lens device 1110 according to the present invention is shown in FIGS. 36 and 37.

The implantable lens device 1110 includes a lens optic portion 1112 and a lens plate haptic portion 1114. The lens plate haptic portion 1114 includes lens plate haptic end portions 1114a, 1114b. The implantable lens device 1110 has the same configuration and arrangement as the implantable lens device shown in FIGS. 33 and 34, except the lens plate haptic portion 1114 is provided with a pair of thin walled bending portions 1115 to enhance and cause concentrated bending of the lens plate haptic portion 1114 at the thin-walled bending zones 1115 to enhance accommodative movement of the implantable lens device 1110. For example, the thin walled bending zones 1115 can be provided by decreasing the thickness of the lens plate haptic portion 1114, as shown in FIG. 37.

An eleventh embodiment of an implantable lens device 1210 according to the present invention is shown in FIG. 38.

The implantable lens device 1210 includes a lens optic portion 1212 and four (4) spaced apart lens plate haptic portions 1214. The lens plate haptic portions 1214 each includes lens plate haptic center portion 1214c connected to a lens plate haptic end portion 1214a. The lens plate haptic center portions 1214c are each provided with a center plate reinforcement 1217 and the lens plate haptic end portions are each provided with end frame type reinforcement 1219a (e.g. H-shaped frame reinforcement). Alternatively, the frame type reinforcements 1219a can be replace with plate type reinforcements. The implantable lens device 1210 can be configured or design to allow most bending to occur at bending zones 1215a (single bending arrangement), or at multiple bending zones 1215a and 1215b (compound bending arrangement with spaced apart bending zones to enhance bending flexibility and providing multiple modes of bending). The lens plate haptic portions 1214 are shown spaced apart and functioning somewhat independently, however, the spacing can be increase or decreased or eliminated. Further, the lens plate haptic portions 1214 can be mechanically coupled (e.g. flexible or elastic connection) to function more dependently (e.g. optional ring-shaped structure 1223 of lens material and/or optional ring-shaped reinforcement connected to lens plate haptic portions 1214 by mechanical connection, layering and/or embedding ring-shaped reinforcement in the lens plate haptic portions 1214) so that the lens plate haptic portions 1214 function somewhat dependently.

A twlefth embodiment of an implantable lens device 1310 according to the present invention is shown in FIGS. 39 and 40.

The implantable lens device 1310 includes a lens optic portion 1312 and a lens plate haptic portion 1314. The lens plate haptic portion 1314 includes lens plate haptic end portions 1314a, 1314b. The lens plate haptic portion 1314 is provided with a ring-shaped structure 1323 (e.g. ring-shaped molded extension or protrusion of lens material) to stiffen and stabilize the lens optic portion 1312. The ring-shaped extension 1323 can also increase the overall thickness of the implantable lens device 1310 to fill the capsular bag when implanted in the capsular bag as an intraocular lens (e.g. ring-shaped molded extension can contact inside of posterior side of the capsular bag in one orientation when implanted in the capsular bag, or if increased in diameter can contact inside of remaining portion of anterior side of the capsular bag after capsularhexis in opposite orientation when implanted in the capsular bag). Alternatively, the ring-shaped protrusion can extend through the opening in the capsular bag and provide centering therein when initially implanted in the capsular bag. In the application of a phakic refractive lens, the ring-shaped protrusion can provide vaulting of the lens optic portion 1312 over the iris or natural crystalline lens to prevent inadvertent contact of the lens optic portion 1312 with the center portion of the natural crystalline lens so as to prevent cataract formation on the surface of the center portion of the natural crystallline lens. The ring-shaped extension 1323 can be provided with an optional ring-shaped reinforcement to further stiffen and stabilize the lens optic portion 1312. The lens plate haptic portions 1314a, 1314b are provided with frame reinforcements 1319a, 1319b to stiffen and stabilize the lens plate haptic portions 1314a, 1314b, and enhance bending at the connection between the lens plate haptic portions 1314a, 1314b and the lens optic portion 1312. Alternatively, the frame reinforcements can be replace with plate reinforcements. The ring-shaped structure 1623 also provide a moment arm to enhance bending between the lens optic portion 1312 and lens plate haptic end portoins 1314a, 134b when a differential pressure is applied across the capsular bag, as an intraocular lens, while compressive or tensile force are applied simulataneously on the edges of the lens plate haptic portion 1314.

An alternative embodiment of the implantable lens device 1310 shown in FIGS. 39 and 40, is shown as implantable lens device 1310′ in FIG. 41. In this embodiment, another ring-shaped structure 1633 is provided on an opposite side of the implantable lens device 1310′. This arrangement further fills the capsular bag (intraocular lens application), or prevents contact of the lens optic portion 1312 with eye tissue on both sides of the implantable lens device 1310 (e.g. center portion of natural crystalline lens and edge of pupil through iris in phakic refractive lens application). Again, the diameter, size, shape, orientation, and conformation of the ring-shaped structure 1323 may be changed or adjusted for particular applications. A further alternative embodiment of the implantable lens device 1310″ is shown in FIG. 42. In this embodiment, the lens plate haptic end portions 1314a″, 1314b″ are oriented at an angle relative to the lens optic portion 1312″ and ring-shaped structure 1323″ to facilitate the starting of bending of the lens plate haptic end portions 1314a″, 1314b″ relative to the lens optic portion 1312 during the accommodative movement.

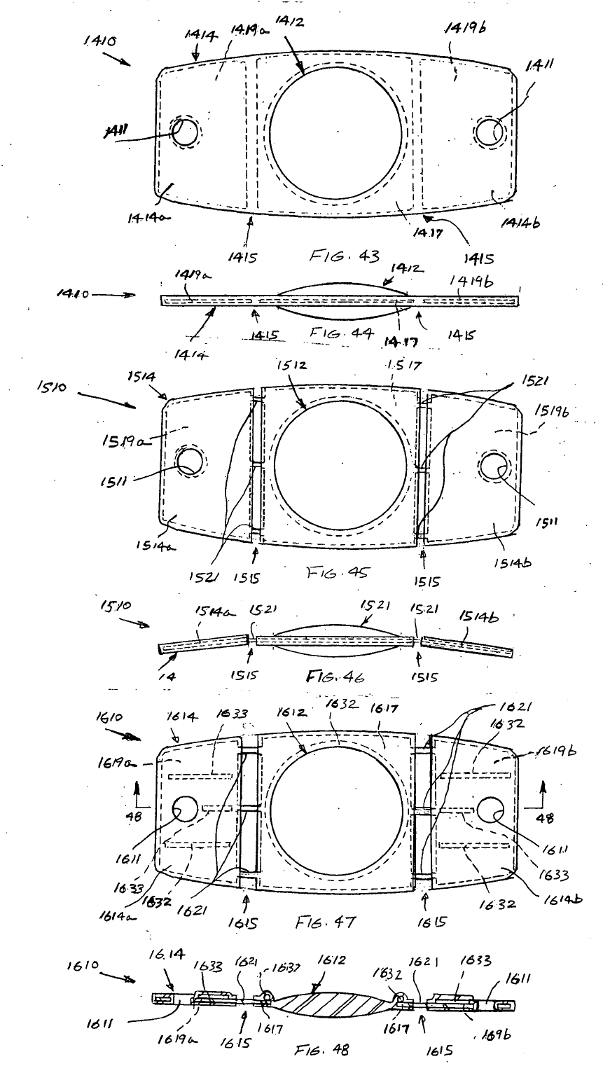

A thirteenth embodiment of an implantable lens device 1410 according to the present invention is shown in FIGS. 43 and 44.

The implantable lens device 1410 includes a lens optic portion 1412 and a lens plate haptic portion 1414. The lens plate haptic portion 1414 includes lens plate haptic end portions 1414a, 1414b. A center portion of the lens plate haptic portion 1414 is provided with a center plate reinforcement 1417, and the lens plate haptic end portions 1414a, 1414a are provided with end plate reinforcements 1419a, 1419b. The center plate reinforcement 1417 strengthens and stabilizes the lens material surrounding the lens optic portion 1412, and the end plate reinforcements 1419a, 1419b strengthen the lens plate haptic end portions 1414a, 1414b. A pair of bending zones 1415 are provided by full thickness lens material (i.e. thickness of lens plate haptic 1414 remains full thickness at these locations), however, the void and spacing between the center plate reinforement 1417 and the end plate reinforcements 1419a, 1419b enhance bending of the lens material at the location of the bending zones 1415. The lens haptic portion 1414 and end plate reinforcements 1419a, 1419b are provided with through holes 1411 to provide an anchoring or securement means with the tissue of the eye adhering through the through holes 1411 during healing.

A fourteenth embodiment of an implantable lens device 1510 according to the present invention is shown in FIGS. 45 and 46.

The implantable lens device 1510 includes a lens optic portion 1512 and a lens plate haptic portion 1514. The lens plate haptic portion 1514 includes lens plate haptic end portions 1514a, 1514b. The implantable lens device 1510 is the same as the implantable lens device 1410 shown in FIGS. 43 and 44, except the center plate reinforcement 1517 is connected to the end plate reinforcements 1519a, 1519b by connecting tabs 1521 (e.g. one, two, three (shown), four, five, six, seven, eight or multiple tabs) and the lens material at the location of the bending zones 1515 is void or discontinuous. Specifically, the lens plate haptic portion is divided into three (3) portions including a center plate haptic portion 1514c and two (2) plate haptic end portions 1514a, 1514b shown as having a substantially uniform thickness. Alternatively, the thickness of plate haptic portions 1514a, 1514b , 1514c can vary in one (1) or two (2) dimensions (i.e. length and width of individual plate haptic portions 1514a, 1514b , 1514c ). The center plate reinforcement 1517 and end plated reinforcements 1519a, 1519b are preferably made from a single piece of material (e.g. polyimide sheet cut with a computer controlled waterject to the shape with the connecting tabs shown). The bending of the plate haptic portion 1514 is enhanced by removing the lens material at the bending zones 1515, and allowing the connecting tabs 1521 only to be subjected to the entire load or bending forces at these specific locations. The substantially reduce thickess of the plate reinforcements 1517 and 1519a, 1319b verses the thickness of the lens material of the lens plate haptic 1514 greatly enhances bending of the connection tabs 1521 by providing a moment arm due to the thickness differential between the lens plate haptic portion 1514 and the thinner plate reinforcements 1517 and 1519a, 1519b . Specifically, shear forces exerted on the surface of the lens platic haptic portion 1514 during compression or tension of the implantable lens device acting on at least a portion of the length of the thickness differential (e.g. one-half thickness differential) enhances the bending moment on the connecting tabs 1521 adding to the bending forces concentrated on the connecting tabs 1521 enhancing bending or other deformation thereof (e.g. compressive or tension force may also shorten or lengthen connecting tabs 1521 and overall lens of implantable lens device 1510). This arrangement or modification thereof can possible provide for force multiplication on the connecting tabs 1521 to further enhance bending thereof. For example, instead of locating the plate reinforcements 1517 and 1519a, 1519b centerplane in the thickness dimension of the lens haptic portion 1514, the plate reinforcements are positioned alternating off center from the centerplace (e.g. center plate reinforcement 1517 is relocated or moved closer to front surface of the lens haptic portion 1514 and the end plate reinforcements 1519a, 1519b are relocated or moved closer to back surface of the lens haptic portion 1514 to create moment arms within the off plane layers of the alternating plate reinforcements at the location of the bending zones 1515). Even further, the plate reinforcements can be bend (e.g. z-shaped) at the location of the bending zones to provide the structural compatibility of the plate reinforcement being out of plane or off plane relative to adjacent plate reinforcements.

A fifteenth embodiment of an implantable lens device 1610 according to the present invention is shown in FIGS. 47 and 48.

The implantable lens device 1610 includes a lens optic portion 1612 and a lens plate haptic portion 1614. The lens plate haptic portion 1614 includes lens plate haptic end portions 1614a, 1614b . The implantable lens device 1610 has the same configuration as the implantable lens device 1510 shown in FIGS. 45 and 46, except additional reinforcements 1632, 1633 have been combined with the plate reinforcements 1617, 1619a, 1619b. Specifically, a ring-shaped reinforcement 1632 has be provided around lens optic portion 1612 and layered on top of the center plate reinforcement 1617, and rod-shaped reinforcements 1632, 1633 have been provided in lens plate haptic end portions 1614a, 1614b to further strengthen the separate portions of the lens haptic portion 1614. The additional reinforcement stiffen the separate portions of the lens haptic portion 1614 enhancing bending at the bending zone 1615 by more effectively transmitting and conveying and concentrating the bending forces on the connecting tabs 1621.

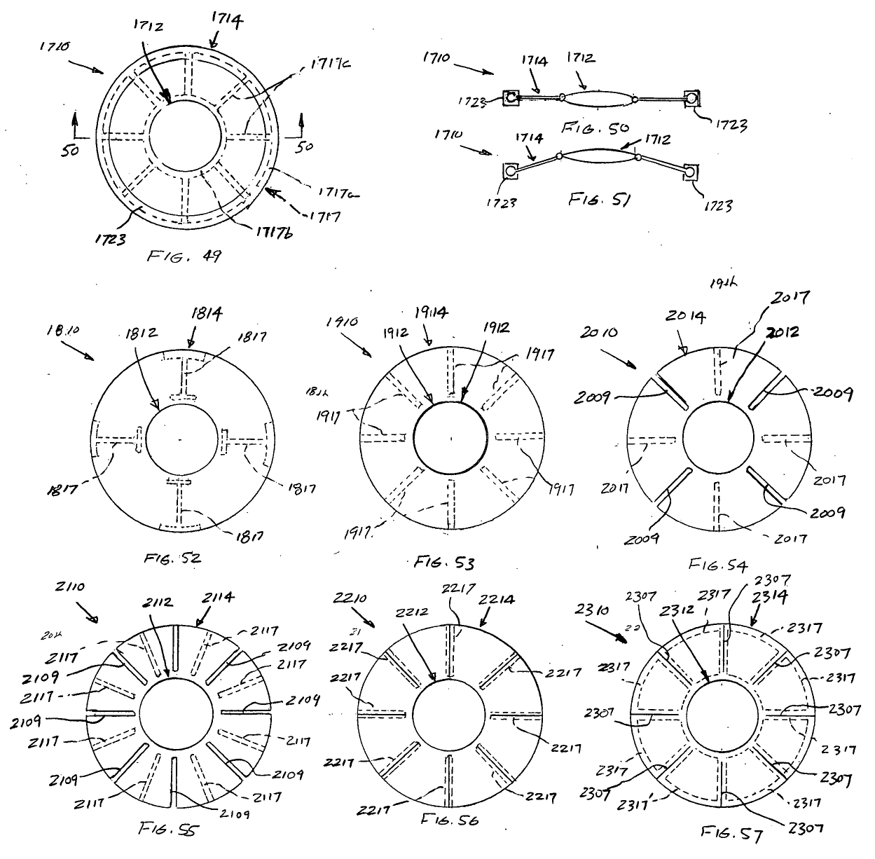

A sixteenth embodiment of an implantable lens device 1710 according to the present invention is shown in FIGS. 49, 50 and 51.

The implantable lens device 1710 includes a lens optic portion 1712 and a round lens plate haptic portion 1714. A ring-shaped structure 1723 is provided at the perimeter of the lens plate haptic portion 1714. The lens plate haptic portion 1714 is provided with a ring-shaped reninforcement 1717 including an outer plate reinforcement ring 1717a and an inner plate reinforcement ring 1717b connected together by radial spoke plate reinforcements 1717c. The ring-shaped reinforcement concentrates bending forces at the ring-shaped connection between the lens optic protion 1712 and lens haptic portion 1714. The ring-shaped structure 1723 can have sharp edges to reduce or eliminate fibrosing of tissue from the outer perimeter towards the center o the implantable lens device 1710. The bending mode of the implantable lens device is shown in FIGS. 50 and 51.

Alternative or modified versions of the implantable lens device 1710 are shown in FIGS. 52 to 57.

In the version shown in FIG. 52, the implantable lens device 1810 is provided with four (4) separate reinforcements 1817 acting somewhat independently. The separate reinforcements 1817 can be H-shaped frame type reinforcements as shown, or alternatively, can be spokes 1917, as shown in FIG. 53, or elongated fan-shaped plate type reinforcements. The implantable lens device 2010 is be provided with radial slots 2009 in combination with radial spokes 2017, as shown in FIG. 54. The slot provide transverse flexibility and accommodates the change in size or surface are of the lens optic portion 2014 during accommodative movement of the lens optic portion 2012. An enhanced version of the implantable lens device 2010 having more slots and spokes is shown as implantable lens device 2110 in FIG. 55.

In the version shown in FIG. 56, the implantable lens device 2210 is provided with a plurality of radial slits 2207. The lens plate haptic portion 2214 is provided with spoke type reinforcements 2217 bridging the slits 2207. The spoke type reinforcements can act as flexible webbing sealing the slits 2207, or alternatively, can also be provided with corresponding partial depth slits or completely through depth slits so that the lens plate haptic portion is divided into a plurality of fan-shaped lens haptic plate portions functioning somewhat independently. Alternatively, the spoke type plate reinforcements are replaced with a plurality of fan-shaped frame type reinforcement(s) 2317, or alternatively plate type, combined with a plurality of slits 2307. The slits 2307 in some embodiments do not extend through, partially extend through, or completely extend through the reinforcement(s) 2317.

A seventeenth embodiment of an implantable lens device 2410 according to the present invention is shown in FIG. 58.

The implantable lens device 2410 includes a lens optic portion 2412 and a lens plate haptic portion 2414. The lens plate haptic portion 2414 includes lens plate haptic end portions 2414a, 2414b. The lens haptic portion 2414 is provided with a plate type reinforcement 2417 having substantially the same size and shape as the lens haptic portion 2414 (excluding the lens optic portion 2412 and through holes 2411). The reinforcement 2417 stiffens the lens plate haptic portion 2414 and implantable lens device 2410. The plate reinforcement 2417 can be replace with a three-dimensional profiled reinforcement having a plate structure combined with sculpting features such as longitudinal ribs, transverse ribs, protrusions, grooves, raised matrix pattern, stringers, and other three dimensional features. The reinforcement 2417 can be one-piece or multiple pieces assembled together. The reinforcement 2417 can be tailored to vary in tensile strength, compressive strength, shear strength, in one (1), two (2), or three (3) or more dimensions depending on the application.

An eighteenth embodiment of an implantable lens device 2510 according to the present invention is shown in FIG. 59.

The implantable lens device 2510 includes a lens optic portion 2512 and a lens plate haptic portion 2514. The lens plate haptic portion 2514 includes lens plate haptic end portions 2514a, 2514b. The lens plate haptic portion 2514 is provided with multiple plate reinforcements 2517a, 2517b, 2517c, 2517d. The spacing between adjacent plate reinforcements increases from the edges of the lens plate haptic portion 2514 to the center of the implantable lens device. Alternatively, the spacing can be uniform, or taper in the opposite direction depending on application.

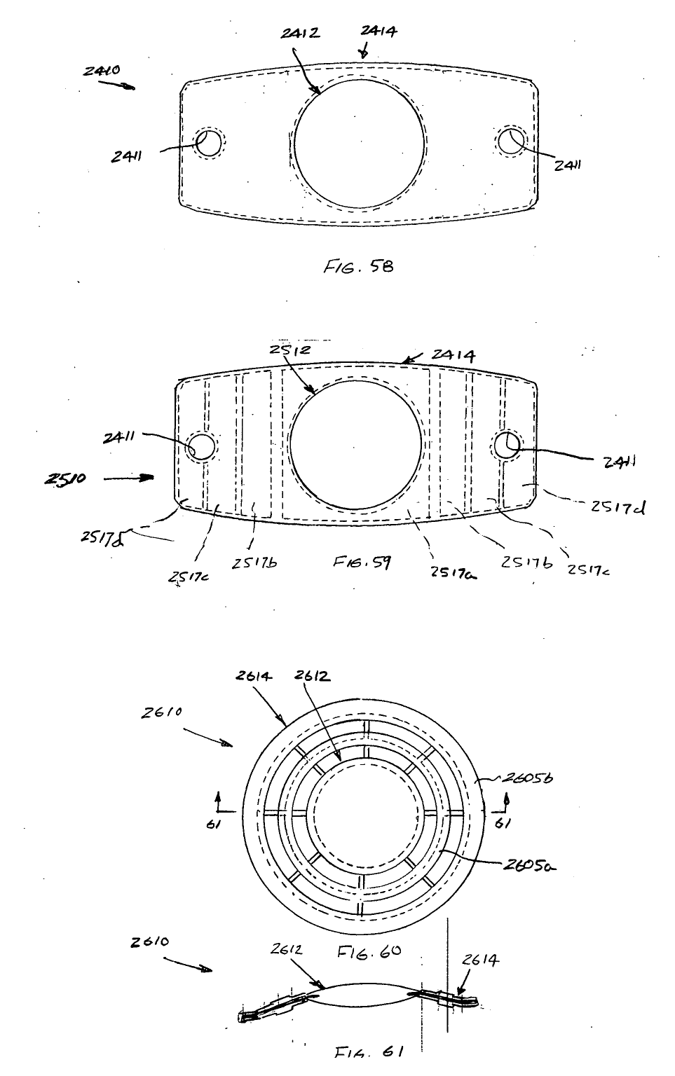

A nineteenth embodiment of an implantable lens device 2610 according to the present invention is shown in FIGS. 60 and 61.

The implantable lens device 2610 includes a lens optic portion 2612 and a round lens plate haptic portion 2614. The lens plate haptic portion 2614 includes a plurality of rings 2605a and 2605b. The number of rings can be increased or decreased depending on the application. A multiple ring type reinforcement 2617 having multiple reinforcement rings 2617a, 2617b, 2617c are provided in the lens optic portion 2612, and lens haptic rings 2605a, 2605b. The reinforcement 2617 included spokes 2617d. The reinforcement can be made as one piece, or multiple pieces. The spokes 2617d can connected or not connected (i.e. independent) or partially connected to the reinforcement rings 2617a, 2617b, 2617c. The lens optic portion 2612, lens haptic rings 2605a, 2605b are spaced apart with ring shape grooves separating same, or alternatively, with thin lens material webbing connecting the components together. For example, a one piece reinforcement 2617 of sheet polyimide is cut with a high pressure waterject to have the configuration shown in FIG. 60. The reinforcement 2617 is then inserted into a mold cavity for a molded lens, and the lens material at least partially embeds the reinforcement. Alternative, a lens optic portion is machined from stock material (e.g. with computer controlled lathe or milling machine), and then the one piece reinforcement is layered, bonded, mechanically connected or otherwise attached to the lens optic portion.

The implantable lens device 2610 can be configured to be highly flexible and essentially free floating as a unit with the capsular bag. The pressure differential on opposite sides of the capsular bag mainly moves and bends the implantable lens device 2610 during accommodative movement while the compressive strength and tensile strength of the reinforcement 2617 enhances accommodative movement.

I claim:

1. An implantable accommodating intraocular lens device, said device comprising:
a lens optic portion made of a lens material;
a lens plate haptic portion including at least two lens plate haptic end portions, said lens plate haptic portion being made of said lens material;
at least one reinforcement provided along the length of each said lens plate haptic end portions, said reinforcement being made of a reinforcement material having a greater strength relative to the lens material to strengthen the said lens optic end portions; and
an unreinforced and highly pliable connection zone located at a connection of each lens plate haptic end portions with said lens optic device, whereby bending forces are applied by eye tissue onto said lens plate haptic end portions and transmitted by said reinforcements to concentrate bending force at said connection zones to substantially bend said lens haptic end portions relative to said lens optic portion.

2. An implantable accommodating intraocular lens device, said device comprising:
a lens optic portion made of a lens material;
a lens plate haptic portion including at least two reinforced lens plate haptic end portions connected to said lens optic portion, said lens plate haptic portion being made of said lens material;
at least one reinforcement provided along at least a portion of the length of each said lens plate haptic end portions, said reinforcement being made of a reinforcement material having a greater strength relative to the lens material to strengthen the said lens optic end portions.

3. An implantable accommodating intraocular lens device, said device comprising:
a lens optic portion made of a lens material;
a lens plate haptic portion connected to said lens optic portion, said lens plate haptic portion made of said lens material;
at least one reinforcement provided along at least a portion of the length of said lens plate haptic portion, said reinforcement being made of a reinforcement material having a greater strength relative to the lens material to strengthen the lens optic portion.

4. A lens device according to claim 1, wherein said lens optic portion is a reinforced lens optic portion provided with at least one reinforcement.

5. A lens device according to claim 1, wherein said lens device is sufficiently pliable along at least one axis to allow insertion through a small incision in the eye.

6. A lens device according to claim 1, wherein said at least one reinforcement is a plate type reinforcement.

7. A lens device according to claim 1, wherein said at least one reinforcement is a rod type reinforcement.

8. A lens device according to claim 1, wherein said at least one reinforcement is a frame type reinforcement.

9. A lens device according to claim 8, wherein said at least one reinforcement is a H-shaped frame type reinforcement.

10. A lens device according to claim 1, wherein said lens device is pre-stressed.

11. A lens device according to claim 1, wherein said lens haptic end portions are pre-stressed.

12. A lens device according to claim 1, wherein said lens optic portion is pre-stressed.

13. A lens device according to claim 4, wherein said reinforcement for said lens optic portion is lens insert is a ring-shaped reinforcement.

14. A lens device according to claim 1, wherein said lens optic portion is provided with a reinforcement located adjacent said bending zones.

15. A lens device according to claim 1, wherein said reinforcement is a matrix type reinforcement.

16. A lens device according to claim 1, wherein said lens device is hingeless.

17. A lens device according to claim 1, wherein said lens haptic end portions are each provided with at least one hinge portion.

18. A lens device according to claim 1, wherein said lens haptic end portions are each provided with a hinge portion at said bending zones.

19. A lens device according to claim 3, wherein said reinforcement is a plate type reinforcement having substantially the same shape and size as said lens haptic poriton.

20. A lens device according to claim 3, wherein said reinforcement is a plate type reinforcement having multiple reinforcement portions connected together by at least one tab located at a bending zone.
